(19) United States
(12) Patent Application Publication
(10) Pub. No.: US 2009/0136624 A1
(43) Pub. Date: May 28, 2009

(54) LACTIC ACID BACTERIA PRODUCING NISIN AT HIGH CONCENTRATION AND METHOD FOR SELECTING THE SAME

(75) Inventors: Naoko TANAKA, Kawasaki-shi (JP); Hiroaki NISHIUCHI, Kawasaki-shi (JP); Akinori UEHARA, Kawasaki-shi (JP); Nobutoshi MATSUMOTO, Kawasaki-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 12/359,677

(22) Filed: Jan. 26, 2009

Related U.S. Application Data
(63) Continuation of application No. 11/508,191, filed on Aug. 23, 2006, now abandoned.
Application No. 11/508,191 is a continuation of application No. PCT/JP2004/018241, filed on Dec. 1, 2004.

(30) Foreign Application Priority Data
Feb. 23, 2004 (JP) 2004-045626

(51) Int. Cl. A23L 3/3463; C12N 1/20; C12N 15/01

(52) U.S. Cl. 426/61; 435/252.9; 435/448; 435/253.6

(57) ABSTRACT

The present invention provides lactic acid bacteria producing Nisin, a method for selecting the same, and foods or feeds using the lactic acid bacteria.

### CROSS-REFERENCE TO RELATED APPLICATIONS

The present application is a continuation of U.S. Ser. No. 11/508,191, filed on Aug. 23, 2006, which is a continuation of PCT/JP2004/018241, filed on Dec. 1, 2004, which claims priority to JP 045626/2004, filed on Feb. 23, 2004, the entire contents of these applications is incorporated herein by reference.

### BACKGROUND OF THE INVENTION

1. Field of the Invention

The present invention provides lactic acid bacteria producing Nisin, a method for selecting the same, and foods or feeds using the lactic acid bacteria.

2. Discussion of the Background

Some Lactococcus lactis (lactic acid bacteria) strains produce lactic acid and Nisin (an antibacterial peptide from sugars by fermentation). Its cells and culture solution have bacteriostatic and antibacterial effects to microorganisms. In recent years, these strains have attracted much interest for its ability to improve food preservation.

Nisin is an antibacterial peptide with a molecular weight of approximately 3.5 kDa comprising 34 amino acids and containing lanthionine, β-methyllanthionine, dehydroaranine and dehydrobutyrin in a molecule. Nisin A, Nisin Z and Nisin Q have been reported to date as natural amino acid substituted substances. It has been known that the antibacterial spectrum thereof is wide and the antibacterial effect is exhibited in not only Gram-positive bacteria but also Gram-negative bacteria (Gill A. O. et al. Adv. Int J Food Microbiol. 2003, Vol. 80, p 25 1-9).

Nisin has been also been approved by the U.S. FDA as only one GRAS substance among bacteriocins, and it is a safe substance which has used wide acceptance in foods, feeds, pharmaceutical preparations and the like.

Examples of using Lactococcus lactis producing Nisin and its culture as a bacteriostatic agent, the use of a culture solution as food additives (JP-A-5-268975) and a method for keeping perishable foods or fermented foods by mixing the same with Lactococcus lactis (JP-A-5-211859) have been reported. Further, Lactococcus lactis producing Nisin has also been reported for use as a mouthwash or pharmaceutical preparation (JP-A-9-077681).

De Vuyst et al. (Blackie Academic and Professional, 1994, p 151-211) report a lactic acid bacteria belonging to Lactococcus lactis having a high producibility of Nisin, which produces 6,750 IU of Nisin per milliliter of a culture solution. However, this reference fails to disclose specific culturing conditions. There is a report, however, that in the batch culturing of Lactococcus lactis UL719, 4,100 IU of Nisin per milliliter of a culture solution is produced (Amiali et al. Adv. World J. Microbiol. Biotecnoly, 1998, p 887-894).

As a method for selecting strains having high Nisin producibility, Qiao et al (Adv. Biotechnol. Let., 1997, p 199-202) report that strains selected using an erythromycin resistance as an index have a resistance to 5,000 IU/mL of Nisin and provide, the highest Nisin producibility which is 10 times that of Lactococcus lactis N8 strain used as a parent strain. It is described that the highest value is 2.8×10⁻⁵ IU/cfu. However, the number of bacteria for a culture solution is not described. Further, the total amount of Nisin production of a culture solution is not indicated. With the exception of Qiao et al, there is no report of a method for selecting strains having a high Nisin producibility, nor is there a report of a method for selecting strains having a high producibility of Nisin using a Nisin resistance as an index.

JP-A-2002-85083 alludes to a method in which Nisin is obtained at a high concentration from a culture solution of lactic acid bacteria having a low Nisin producibility is subjected to membrane concentration, or Nisin is accumulated in a medium by continuous culturing and the like. However, the continuous culturing requires an intricate costly device, and the Nisin activity decrease in the concentration method. Accordingly, these methods are not always efficient methods.

U.S. Pat. No. 5,965,178 reports that the bacteriostatic effect of a Nisin-producing lactic acid bacteria culture to microorganisms is higher in the presence of lactic acid bacteria. Actually, the bacteriostatic effect is increased by a combined use of lactic acid bacteria and a Nisin solution in a method for making miso (JP-A-2001-224359) or a method for making boiled beans (JP-A-2003-116477).

Meanwhile, there remains a problem in the existing art in that bacteria are removed in a method for preparing a Nisin solution at a high concentration by the foregoing concentration or continuous culturing. Accordingly, there remains a critical need for a culture solution containing a high concentration of NisinN and lactic acid bacteria is prepared by a simple method such as batch culturing. To this end, it is effective to develop a method in which lactic acid bacteria producing Nisin at a high concentration are grown and selected and to develop lactic acid bacteria having a high Nisin producibility. When lactic acid bacteria having a high Nisin producibility are developed, a culture thereof is used in foods, drinks and feeds, making it possible to efficiently improve a keeping property of these foods, drinks and feeds.

### SUMMARY OF THE INVENTION

It is an object of the present invention to provide lactic acid bacteria capable of producing a high concentration of Nisin in a culture solution even by simple batch culturing and a culture solution thereof It is also an object of the present invention to provide a method for easily selecting lactic acid bacteria having a high Nisin producibility.

For solving the problems existing in the art and to satisfy the foregoing objects of the present invention, the present inventors have found that lactic acid bacteria having a higher Nisin producibility than what has been so far reported can be obtained even in the batch culturing by selecting bacteria grown with a bacteriocin produced by lactic acid bacteria, especially in a synthetic medium containing a bacteriocin produced by lactic acid bacteria using a resistance to Enterocin or Nisin as an index. Representatively, the present invention embraces the following embodiments:

(1) A lactic acid bacteria which produce 6,800 IU or more of Nisin per milliliter of a supernatant of a medium in batch culturing with a liquid medium.

(2) The lactic acid bacteria of (1), which produce 8,100 IU or more of Nisin per milliliter of the supernatant in the medium.

(3) The lactic acid bacteria of (1), which produce 10,125 IU or more of Nisin per milliliter of the supernatant in the medium.

(4) The lactic acid bacteria of (1), wherein the liquid medium comprises yeast extract, sodium chloride, glucose, and calcium carbonate.

(5) The lactic acid bacteria of (4), wherein the liquid medium contains 0.5% yeast extract, 0.5% sodium chloride, 3% glucose and 1.5% calcium carbonate.

(6) The lactic acid bacteria of (4), wherein the liquid medium further comprises serine and cysteine.

(7) The lactic acid bacteria of (1), wherein Nisin produced is Nisin A.

(8) The lactic acid bacteria of (1), wherein Nisin produced is Nisin Z. (9) The lactic acid bacteria of (1), wherein said lactic acid bacteria belong to the genus Lactococcus.

(10) The lactic acid bacteria of (9), wherein said lactic acid bacteria belong to the sub-species Lactococcus lactis ssp. Lactis.

(11) The lactic acid bacteria of (9), wherein said lactic acid bacteria is Lactococcus lactis AJ110212 (FERM BP-8552).

(12) A method for selecting Nisin-producing lactic acid bacteria which produce 6,800 IU or more of Nisin per milliliter of a supernatant of a medium in batch culturing with a liquid medium, which comprises
growing a Nisin-producing lactic acid bacteria in a synthetic medium containing a bacteriocin produced by lactic acid bacteria at a pH ranging from 6.0 to 7.0 at a temperature ranging from 25° C. to 35° C. for a time sufficient to grow the Nisin-producing lactic acid bacteria; and
selecting Nisin-producing lactic acid bacteria which produce 6,800 IU or more of Nisin per milliliter of a supernatant of a medium in batch culturing with a liquid medium.

(13) The method of (12), wherein after said growing said method further comprises
inducing spontaneous mutation or mutation with a mutation inducer or ultraviolet rays to produce a mutant strain;
plating the mutant strain on synthetic agar medium containing one or more bacteriocin; and
growing said mutant strain on said synthetic agar medium for a time and under conditions suitable to grow said mutant strain.

(14) The method of (12), wherein the bacteriocin is Enterocin or Nisin.

(15) The method of (12), wherein the bacteriocin is Nisin in an amount of from 11,000 to 90,000 IU per milliliter of the medium.

(16) The method of (12), wherein the bacteriocin is Nisin in an amount of from 20,000 to 80,000 IU per milliliter of the medium.

(17) A culture solution containing lactic acid bacteria which is obtained by culturing the lactic acid bacteria of (1) in the medium.

(18) A food, drink, or feed comprising the 0.01 to 10% of the culture solution according to (17).

(19) A dry product of the culture solution of (17), wherein said culture solution is spray-dried or drum-dried.

(20) A food, drink, or feed comprising the 0.01 to 10% of the dry product of the culture solution according to (19).

(21) A supernatant of the culture solution of (17), wherein said lactic acid bacteria are removed from said culture solution.

(22) A food, drink, or feed comprising the 0.01 to 10% of the supernatant of the culture solution according to (21).

(23) A dry product of the supernatant of the culture solution according to (21), wherein said supernatant is spray-dried or drum-dried.

(24) A food, drink, or feed comprising the 0.01 to 10% of the supernatant of the culture solution according to (23).

The above objects highlight certain aspects of the invention. Additional objects, aspects and embodiments of the invention are found in the following detailed description of the invention.

### DETAILED DESCRIPTION OF THE INVENTION

Unless specifically defined, all technical and scientific terms used herein have the same meaning as commonly understood by a skilled artisan in biochemistry, molecular biology, and foods.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified. The present invention provides lactic acid bacteria producing Nisin, a method for selecting the same, and foods or feeds using the lactic acid bacteria.

The lactic acid bacteria of the present invention are strains that have a resistance to a bacteriocin produced by lactic acid bacteria and produce Nisin in an amount of 6,800 IU or more per milliliter of a supernatant of a medium in batch culturing with a liquid medium containing 0.5% yeast extract, 0.5% sodium chloride, 3% glucose and 1.5% calcium carbonate.

An embodiment of the present invention provides a method for obtaining a culture of lactic acid bacteria as described below.

The liquid medium is not particularly limited so long as it is capable of facilitating and maintaining growth of lactic acid bacteria that produce Nisin. Generally the growth medium is a synthetic medium made of an aqueous solution of a sugar source, a nitrogen source, inorganic salts and the like. This synthetic medium preferably contains at least one sugar source and at least one nitrogen source. As a sugar source, monosaccharides, such as glucose and galactose, lactose and sucrose are preferred. As a nitrogen source protein hydrolyzate, peptone, yeast extract, fish meat extract, casamino acid, malt juice and the like are preferred. The growth medium also preferably contains at least one inorganic salt for example: sodium chloride, calcium carbonate and the like. A liquid medium containing 0.5% yeast extract, 0.5% sodium chloride, 3% glucose and 1.5% calcium carbonate is preferable. The pH of the medium is preferably adjusted to range from 6.0 to 7.0. Further, the medium should be sterilized and then cooled prior to use. To adjust the pH to from 6.0 to 7.0 during the culturing is important for production of Nisin, and calcium carbonate is preferably added as needed to maintain the pH within the desired range.

As used herein, the term “lactic acid bacteria” refers to Lactococcus lactis strains. Preferably, the Lactococcus lactis strains of the present invention produce lactic acid and Nisin (an antibacterial peptide from sugars by fermentation). The lactic acid bacteria producing Nisin preferably belong to Lactococcus lactis ssp. lactis, and examples thereof include JCM 7638, ATCC 11454, NCDO 497, IFO 12007 and the like.

In the method of the present invention, the medium is inoculated with the lactic acid bacteria producing Nisin produced at a concentration of from 10⁵ to 10⁹ cells/mL. Culturing is conducted at a temperature ranging from 25° C. to 35° C., preferably from 27.5° C. to 32.5° C., with stirring at a low speed of from 0 (allowed to stand still) to 150 rpm while adjusting pH to from 5.0 to 6.5, preferably 5.5.

The Nisin activity of a culture solution is measured by the Ishizaki et al method (Adv. J. Fac. Agr., Kyushu Univ., 40, p 73-85). Specifically, the Nisin activity in the resulting culture solution is measured by HPLC. A Nisin A pharmaceutical preparation 30 (Sigma) is used as a standard. At this time, a value of an activity of Nisin is 40 IU/μg prescribed in international units. When preparing samples for measurement of the Nisin activity, Tween-20 is added to a sampled culture solution such that a final concentration becomes 0.1%, and subsequently the sample is mixed well. The mixture is centrifuged or treated through a filter of 0.22 μm to remove the lactic acid bacteria.

A method for obtaining lactic acid bacteria producing Nisin at a high concentration is described below. In the method, mutation of a strain is used. First, lactic acid bacteria are cultured in a synthetic liquid medium capable of growing the same. Subsequently, a part of the culture solution of lactic acid bacteria is inoculated in a liquid medium containing a bacteriocin such as Plantaricin S, Herveticin J, Pediocin PA-1 or Enterocin, preferably in a liquid medium containing a bacteriocin producing lactic acid bacteria, such as Plantaricin S, Herveticin J, Pediocin PA-1 or Enterocin, more preferably in a liquid medium containing lactibiotics (a classification of bacteriocins produced by lactic acid bacteria) such as Lacticin 481, Lactocin S and Nisin or Class II-type bacteriocins (a classification of bacteriocins produced by lactic acid bacteria) such as Pediocin PA-1 and Enterocin, further preferably in a liquid medium containing Nisin classified in lantiobiotics or Enterocin classified in Class II-type bacteriocins, especially preferably in a liquid medium containing an amount of Nisin is larger than an amount of Nisin produced by a parent strain, and it is grown at 30° C., followed by induction of spontaneous mutation or mutation with a mutation inducer, ultraviolet rays or the like. Examples of the mutation inducer includes N-methyl-N′-nitro-N-nitroguanidine (NTG), ethyl methanesulfonate (EMS), sodium 4-dimethylaminobenzenediazosulfonate (DAPA) and the like.

The mutation induced strain is plated on a synthetic agar medium containing a bacteriocin such as Nisin. At this time, the Nisin concentration of the synthetic agar medium is from 11,000 to 90,000 IU/mL, preferably from 20,000 to 80,000 IU/mL. As a bacteriocin other than Nisin, lactobiotics or Class II-type bacteriocins are plated. The amount of bacteriocin is an amount in which growth of a parent strain can be suppressed, and it is preferably from 2 to 200 times, more preferably from 5 to 200 times. For example, in case of Enterocin, although the amount varies with a parent strain, it can be generally 0.5 μg/mL or more, from 1 to 100 μg/mL, from 2.5 to 100 μg/mL, and from 10 to 100 μg/mL in view of the results reported by Fujita et al (“Bacteriocin produced by Enterococcus faecium TUA 1344L” announced in 2004 Nihon Nyusankin Gakkai).

The number of lactic acid bacteria spread in a medium is preferably such a number that colonies formed are not contacted with one another. Specifically, the number of colonies is preferably from 100 to 300 per plate. For efficiently selecting a mutant with an improved Nisin producibility, a strain whose growth is more efficient than that of a parent strain is selected from among strains grown in the medium. The condition of the growth is observed visually.

The selected strain is cultured according to the foregoing method for obtaining the culture solution of lactic acid bacteria. A microplate assay is conducted using the culture solution, and a strain whose antibacterial activity to index bacteria is higher than that of a parent strain is selected.

The microplate assay is described as follows. To each well of a microplate, a solution obtained by stepwise diluting a supernatant of lactic acid bacteria culture solution and a culture solution of Gram-positive bacteria sensitive to Nisin as an index are added such that the cell number is from 10² to 10⁵ cells/mL. The sample is then mixed. Examples of the index bacteria used at this time include Bacillus subtilis JCM1465T, Lactobacillus sakei JCM1157T and the like. Subsequently, the microplate is incubated at 37° C. for at time ranging from 4 to 24 hours, preferably from 12 to 21 hours. The antibacterial activity, namely the Nisin activity in the culture solution is measured in terms of a degree of growth of the index bacteria. The degree of growth is estimated via an extent of a turbidity (Abs=595 nm) of the well. When the Nisin activity in the culture solution is high to inhibit the growth of the index bacteria, the turbidity of the well is not increased. The parent strain is compared with the mutant strain in the maximum dilution rate of the supernatant of the culture solution with the turbidity of the well increased, and the strain whose dilution rate is higher than that of the parent strain is selected upon estimating that it has a possibility of producing Nisin in an amount which is greater than that provided by the parent strain.

Finally, the above-selected strain is cultured, and the amount of Nisin in the culture solution is measured by HPLC to confirm that the Nisin activity in the culture solution, namely the Nisin producibility of lactic acid bacteria, is increased.

By the foregoing method, lactic acid bacteria capable of producing a high concentration of Nisin in a culture solution, even by batch culturing, can efficiently be obtained. In past reports, the maximum amount of Nisin produced is 6,750 IU/mL (De Vuyst et. al.). Meanwhile, lactic acid bacteria having a higher Nisin producibility than lactic acid bacteria having the highest Nisin producibility among the known examples of the same, namely lactic acid bacteria having a Nisin producibility of 6,800 IU or more per milliliter of the supernatant, can be obtained by culturing under optimum conditions in a liquid medium containing 0.54% yeast extract, 0.5% sodium chloride, 3% glucose and 1.5% calcium carbonate while maintaining pH. Specifically, it is possible to obtain lactic acid bacteria producing at least 7,425 IU/mL of Nisin per milliliter of a supernatant which is a Nisin producibility of 1.1 times the highest Nisin producibility of the known lactic acid bacteria, lactic acid bacteria producing at least 8,100 IU/mL of Nisin per milliliter of a supernatant which is a Nisin producibility of 1.2 times the highest Nisin producibility of the known lactic acid bacteria, lactic acid bacteria producing at least 10,125 IU/mL of Nisin per milliliter of a supernatant which is a Nisin producibility of 1.5 times the highest Nisin producibility of the known lactic acid bacteria, and lactic acid bacteria producing at least 12,150 IU/mL of Nisin per milliliter of a supernatant which is a Nisin producibility of 1.8 times the highest Nisin producibility of the known lactic acid bacteria. It is considered that Nisin in the highest amount of 20,000 IU per milliliter of a supernatant can be obtained by culturing lactic acid bacteria having a Nisin producibility in a more appropriate medium to which serine and cysteine are added or the like.

Lactic acid bacteria having a high Nisin producibility and a culture solution containing the lactic acid bacteria can efficiently be produced by batch culturing in a medium suitable for growth of lactic acid bacteria. An example of such a medium is YDCS medium (0.54% yeast extract, 0.5% sodium chloride, 3% glucose, 0.67 mg/dl serine, 0.67 mg/dl cysteine and 1.5% calcium carbonate). A dry product of the culture solution containing the lactic acid bacteria can be formed by, for example, spray-drying or drum-drying the culture solution containing lactic acid bacteria. Further, a supernatant of the culture solution from which lactic acid bacteria are removed can be formed by, for example, filter treatment or decantation of the culture solution containing the lactic acid bacteria, and a dry product of the supernatant of the culture solution can be formed by, for example, spray-drying or drum-drying the supernatant of the culture solution.

The culture solution, dry-product of the culture solution, supernatant of the culture solution, or dry-product of the supernatant of the culture solution obtained by the foregoing method can increase shelf life of foods, drinks or feeds when added in an amount of from 0.01 to 10%. With respect to addition to foods, drinks, or feeds, the “culture” may be the culture solution as such, the sterilized culture solution, the culture solution with the bacteria removed, or a concentrate or dry product thereof. Examples of foods and drinks include juices, dairy products, meat products, picked products, fermented food seasonings such as miso and soy sauce, and the like. Examples of feeds include a silage and the like. For example, contamination of undesirable microorganisms such as bacteria of the genus Bacillus can be inhibited more effectively than ever by sprinkling the Nisin-producing lactic acid bacteria and the culture solution containing the high concentration of Nisin during a step of making koji in the production of miso or soy sauce. Accordingly, products organoleptically excellent with less microbial contamination are easily formed. Moreover, the microbial contamination of koji can be inhibited by using the foregoing culture solution in koji. It is also possible to make new seasonings by subjecting koji to saltless decomposition and decomposing proteins to a high extent.

When the Nisin-containing culture solution is used as a food bacteriostatic agent, it is considered that the medium ingredients have an adverse organoleptic effect on foods used. However, the use of the Nisin-containing culture solution decreases the addition amount of the culture solution to eliminate the adverse organoleptic effect by the medium ingredients. In producing cheese, there is an example in which Nisin-producing lactic acid bacteria are inoculated as a starter for inhibiting growth of Clostridium bacterium which makes cheese porous. When the lactic acid bacteria produced high Nisin, the bacteriostatic effect thereof is naturally increased, so that cheese without comtamination can be produced with more confidence and reproducibility.

By selecting bacteria grown in a bacteriocin-containing synthetic medium using resistance of lactic acid bacteria to a bacteriocin produced by lactic acid bacteria as an index, lactic acid bacteria having a Nisin producibility of 6,800 IU or more per milliliter of a supernatant of the medium can be obtained in the growth by batch culturing in a liquid medium. When the resulting culture is used in foods or feeds, preservation of foods or feeds can be increased.

Lactic acid bacteria that produce high Nisin can be obtained by the method of the present invention. Accordingly, the lactic acid bacteria of the present invention can produce Nisin at a high concentration. Therefore, a culture solution having a high Nisin activity can be prepared by the simple batch culturing. This culture solution is used in various foods, drinks and feeds so as to be able to improve the preservation of foods, drinks and feeds. Accordingly, the present invention finds tremendous utility in the fields of foods and feeds industry.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Example 1—

A Nisin Z-producing strain (parent strain shown in Table 3) separated from its native environment was plated on M17 medium (manufactured by Difco) prepared such that a Nisin concentration became 1,000, 2,000, 5,000 or 10,000 IU/mL, and was cultured at 30° C. for 2 days. This strain grew in M17 medium having a Nisin concentration of 1,000 or 2,000 IU/mL, but not in M17 medium having a Nisin concentration of 5,000 or 10,000 IU/mL. From the results, it was confirmed that the minimum growth inhibitory concentration (MIC) of this strain to Nisin was 5,000 IU/mL.

The Nisin Z-producing lactic acid bacteria separated from its native environment were precultured in M17 medium, and the lactic acid bacteria were treated with 500 μg/mL of NTG to induce mutation.

The mutated strain was plated on M17 medium, which was prepared such that the Nisin concentration became 1,000, 2,000, 4,000, 8,000, 10,000, 20,000, 40,000, 80,000 or 100,000 IU/mL, and was incubated at 30° C. around 1-3 days.

The number of cells grown in the medium having each Nisin concentration is shown in Table 1.

TABLE 1
Number of cells in which Nisin Z-producing lactic acid
bacteria are grown in a Nisin-containing medium

| Nisin | | | | | |
|---|---|---|---|---|---|
| conc. | | | | | |
| (IU/ml) | 0 | 1,000 | 2,000 | 4,000 | 8,000 |
| Growth | 1.20E+09 | 1.20E+09 | 1.20E+09 | 1.20E+09 | 7.00E+08 |
| number | | | | | |
| (cell/ml) | | | | | |
| Nisin | | | | | |
| conc. | | | | | |
| (IU/ml) | 10,000 | 20,000 | 40,000 | 80,000 | 100,000 |
| Growth | 1.14E+08 | 3.21E+07 | 9.35E+05 | 1.16E+03 | 4.76E+02 |
| number | | | | | |
| (cell/ml) | | | | | |

As shown in Table 1, at the Nisin concentration of 8,000 IU/mL or more, the number of cells capable of growth in the plate started to decrease. Accordingly, it was determined that lactic acid bacteria producing Nisin at a high concentration could be selected in the synthetic medium containing from 8,000 IU/mL to 100,000 IU/mL of Nisin. Thus, 300 strains were selected from each plate, and strains which Nisin producibility was greater than that of the parent strain were selected by microplate assay.

Each strain of the selected lactic acid bacteria was cultured in a Thioglycolate medium without glucose (manufactured by Difco) at 37° C. for 24 hours. The culture solution thereof was seeded with 50 mL of YD medium (0.5% yeast extract, 0.5% sodium chloride, 3.0% glucose and 1.5% calcium carbonate, pH 7.0, Sakaguchi flask), and shaken at 100 rpm to conduct batch culturing.

The amount of Nisin produced by each strain was measured by HPLC. As a result, the number of strains producing Nisin in an amount of 6,800 IU or more per milliliter of the medium is shown in Table 2, and the Nisin activity of the mutant strain obtained in each plate is shown in Table 3.

TABLE 2
Number of strains producing Nisin at a high concentration

| Nisin concentration of medium (IumL) | 8,000 | 10,000 | 20,000 | 40,000 | 80,000 | 100,000 |
|---|---|---|---|---|---|---|
| Number of mutant strains producing | 0 | 0 | 2 | 19 | 5 | 0 |
| 6,800 IU/mL or more of Nisin | | | | | | |

TABLE 3
Nisin activity of strains obtained

| | | | | O.D. of culture |
|---|---|---|---|---|
| | Nisin activity | Lactic acid | Consumed glucose | solution |
| | (IU/mL) | concentration (%) | concentration (%) | (Abs = 595 nm) |
| L. lactis JCM7638 | 3,620 | 1.97 | 2.67 | 5.88 |
| Parent strain | 4,030 | 2.00 | 3.00 | 5.95 |
| L. lactis #404 | 7,243 | 2.15 | 2.33 | 5.94 |
| L. lactis AJ110212 | 12,247 | 2.05 | 2.32 | 6.02 |
| L. lactis #N139 | 10,090 | 1.49 | 2.20 | 4.59 |
| L. lactis #N43 | 8,198 | 1.99 | 3.00 | 5.54 |
| L. Lactis #N113 | 7,483 | 1.94 | 3.00 | 4.27 |
| L. lactis #N84 | 8,413 | 2.12 | 2.71 | 6.63 |

L. lactis JCM7638: general Nisin Z strain
L. lactis #404: strain selected in a 20,000 IU/mL Nisin plate
L. lactis AJ110212: strain selected in a 40,000 IU/mL Nisin plate
L. lactis #N139: strain selected in a 40,000 IU/mL Nisin plate
L. lactis #N43: strain selected in a 40,000 IU/mL Nisin plate
L. lactis #N113: strain selected in a 40,000 IU/mL Nisin plate
L. lactis #N84: strain selected in a 80,000 IU/mL Nisin plate

Consequently, it was determined that strains producing Nisin in an amount of 6,800 IU/ml or more can be efficiently selected in the medium containing Nisin in an amount of from 20,000 IU/mL to 80,000 IU/mL.

L. lactis AJ1 10202 strain having the highest Nisin producibility was obtained from strains selected in the 40,000 IU/mL Nisin-containing plate. L. lactis AJ1 10212 strain has been determined to have the Nisin producibility which is approximately 3.0 times that of the parent strain and approximately 3.4 times that of L. lactis JCM7638 strain, general Nisin Z strain. It has been also determined to have the Nisin producibility which is approximately 1.8 times the highest Nisin producibility, 6,750 IU/mL described in the De Vuyst et al document among the past reports. By the way, L. lactis AJ110212 strain was deposited under deposit number FERM BP-8552 on Nov. 19, 2003 in International Patent Organism Depositary, National Institute of Advanced Industrial Science and Technology.

### Example 2—

An Enterocin-containing solution was prepared as follows. The Enterocin-producing bacterium, Enterococcus faecium JCM 5804 was cultured in MRS medium (manufactured by Difco) at 37° C. for 22 hours with agitation. The culture solution was centrifuged, and the supernatant was then filtered using a filter (Disposable Syringe Filter Unit manufactured by ADVANTEC, Dismic-25cs, Cellulose Acetate 0.45 μm). The supernatant was coarsely purified using an ultrafiltration membrane. Specifically, centrifugation and ultrafiltration (desalting) were conducted to collect the supernatant fraction. In this step, Enterocin in the culture solution was concentrated to prepare a solution containing approximately 200 μg/mL of Enterocin. The antibacterial activity before and after the concentration was confirmed by the spot-on-lawn method using Nisin Z-producing lactic acid bacteria isolated from the natural world as used in Example 1.

The Nisin Z-producing lactic acid bacteria (parent strain shown in Table 3) separated from the natural world were precultured in M17 medium (manufactured by Difco), and the bacteria grown were treated with 500 μg/mL of NTG to induce mutation.

The mutated strain was plated on M17 medium or M17 medium prepared such that the Enterocin concentration became approximately 20 μg/mL, and was incubated at 30° C. around 1-3 days. The number of colonies grown on the M17 plate containing Enterocin was 1/100 compare with in M17 plate without Enterocin. Accordingly, in view of the results in the Nisin-containing M17 medium, it was considered that lactic acid bacteria producing a high concentration of Nisin could be sorted out in the M17 medium containing Enterocin at this concentration. Thus, 108 strains were selected, and strains having the Nisin producibility which was higher than that of the parent strain were selected by microplate assay.

Each strain of the lactic acid bacteria selected was cultured in a Thioglycolate medium without glucose (manufactured by Difco) at 37° C. for 24 hours. The culture solution 25 thereof was seeded in 50 mL of YD medium (0.5% yeast extract, 0.5% sodium chloride, 3.0% glucose and 1.5% calcium carbonate, pH 7.0, Sakaguchi flask), and shaken at 100 rpm to conduct batch culturing.

The amount of Nisin produced by each strain was measured by HPLC. Consequently, 24 strains producing Nisin at a concentration of 6,800 IU or more per milliliter of the medium were obtained.

From the strains selected in the Enterocin-containing plate in this method, L. lactis AJ110376 strain (Nisin activity 10,802 IU/mL) having the highest Nisin producibility was obtained. It was determined that L. lactis AJ110376 strain has a Nisin producibility which is approximately 2.7 times that of the parent strain and approximately 3.4 times that of L. lactis JCM7638 strain, general Nisin Z strain. Further, it was determined that this strain has a Nisin producibility which is approximately 1.6 times the highest Nisin producibility, 6,750 IU/mL described in the De Vuyst et al document among the past reports.

### Comparative Example 1—

L. lactis #N43 described in Example 1 was plated on M17 medium prepared such that the erythromycin concentration became from 0.01 to 0.2 μg/mL, and was incubated at 30° C. around 1-3 days. Subsequently, lactic acid bacteria were cultured in a Thioglycolate medium without glucose (manufactured by Difco) at 37° C. for 24 hours. The culture solution was seeded in YD medium (0.5% yeast extract, 0.5% sodium chloride, 3.0% glucose and 1.5% calcium carbonate, pH 7.0, Sakaguchi flask), and cultured at 100 rpm while being shaken. The amount of Nisin produced by each strain in the supernatant of the medium was measured by HPLC, and the results are shown in Table 4.

TABLE 4
Selection of Nisin-producing bacteria using
an erythromycin resistance as an index

| | | | Consumed |
|---|---|---|---|
| | Nisin activity | Lactic acid | glucose |
| | (IU/mL) | conc. (%) | conc. (%) |
| Parent strain (#N43) | 8198 | 0.00 | 1.99 |
| L. lactis E1 | 7096 | 2.99 | 2.55 |
| L. lactis E2 | 3954 | 3.00 | 2.46 |
| L. lactis E3 | 4713 | 3.00 | 2.25 |
| L. lactis E4 | 5070 | 2.96 | 2.21 |
| L. lactis E5 | 5637 | 3.00 | 2.47 |
| L. lactis E6 | 6804 | 3.00 | 2.41 |
| L. lactis E7 | 4446 | 2.88 | 1.31 |
| L. lactis E8 | 5340 | 2.84 | 1.56 |
| L. lactis E9 | 5979 | 2.93 | 1.87 |
| L. lactis E10 | 4174 | 2.84 | 1.33 |
| L. lactis E11 | 5386 | 3.00 | 2.68 |
| L. lactis E12 | 4613 | 2.97 | 2.25 |
| L. lactis E13 | 4348 | 3.00 | 2.30 |
| L. lactis E14 | 5594 | 2.99 | 2.44 |
| L. lactis E15 | 5147 | 2.87 | 1.45 |
| L. lactis E16 | 4854 | 2.89 | 1.70 |
| L. lactis E17 | 4645 | 2.86 | 1.31 |
| L. lactis E18 | 5042 | 2.99 | 1.84 |
| L. lactis E19 | 7442 | 3.00 | 2.50 |
| L. lactis E20 | 4369 | 2.87 | 1.28 |

As shown in Table 4, a strain which Nisin producibility was improved as compared with the parent strain was not obtained, and the Nisin producibility could not be improved by the selection with the erythromycin resistance as an index described in the Qiao et al document. Consequently, the selection method using Nisin as an index is considered to be by far more efficient than the selection method using the erythromycin resistance as an index.

### Comparative Example 2—

The Nisin Z-producing lactic acid bacteria (parent strain in Table 3) separated from its native environment as described in Example 1 were precultured in M17 medium (manufactured by Difco), and the bacteria grown were treated with 500 μg/mL of NTG to induce mutation.

The mutated strain was plated on Ml 7 medium prepared such that the erythromycin concentration became 0.2 μg/mL, and was incubated at 30° C. around 1-3 days. Subsequently, 20 strains were randomly selected from the lactic acid bacteria grown on the plate. Each of the strains was cultured in a Thioglycolate medium without glucose (manufactured by Difco) at 37° C. for 24 hours. The culture solution was seeded in 50 mL of YD medium (0.5% yeast extract, 0.5% sodium chloride, 3.0% glucose and 1.5% calcium carbonate, pH 7.0, Sakaguchi flask), and cultured at 100 rpm while being shaken. The amount of Nisin produced by each strain in the supernatant of the culture was measured by HPLC. Consequently, a strain having a Nisin activity which was higher than that of the parent strain could not be obtained.

Thus, a strain which Nisin producibility was better than that of the parent strain was not obtained, and the Nisin producibility could not be improved by the selection with the erythromycin resistance as an index described in the Qiao et al document. Accordingly, the selection method with Nisin or bacteriocins other than Nisin as an index is considered to be by far more efficient than the selection method with the erythromycin resistance as an index.

Numerous modifications and variations on the present invention are possible in light of the above teachings. It is, therefore, to be understood that within the scope of the accompanying claims, the invention may be practiced otherwise than as specifically described herein.

1. A lactic acid bacteria which produce 6,800 IU or more of Nisin per milliliter of a supernatant of a medium in batch culturing with a liquid medium.

2. The lactic acid bacteria as claimed in claim 1, which produce 8,100 IU or more of Nisin per milliliter of the supernatant in the medium.

3. The lactic acid bacteria as claimed in claim 1, which produce 10,125 IU or more of Nisin per milliliter of the supernatant in the medium.

4. The lactic acid bacteria as claimed in claim 1, wherein the liquid medium comprises yeast extract, sodium chloride, glucose, and calcium carbonate.

5. The lactic acid bacteria as claimed in claim 4, wherein the liquid medium contains 0.5% yeast extract, 0.5% sodium chloride, 3% glucose and 1.5% calcium carbonate.

6. The lactic acid bacteria as claimed in claim 4, wherein the liquid medium further comprises serine and cysteine.

7. The lactic acid bacteria as claimed in claim 1, wherein Nisin produced is Nisin A.

8. The lactic acid bacteria as claimed in claim 1, wherein Nisin produced is Nisin Z.

9. The lactic acid bacteria as claimed in claim 1, wherein said lactic acid bacteria belong to the genus Lactococcus.

10. The lactic acid bacteria as claimed in claim 9, wherein said lactic acid bacteria belong to the sub-species Lactococcus lactis ssp. Lactis.

11. The lactic acid bacteria as claimed in claim 9, wherein said lactic acid bacteria is Lactococcus lactis AJ110212 (FERM BP-8552).

12. A method for selecting Nisin-producing lactic acid bacteria which produce 6,800 IU or more of Nisin per milliliter of a supernatant of a medium in batch culturing with a liquid medium, which comprises
growing a Nisin-producing lactic acid bacteria in a synthetic medium containing a bacteriocin produced by lactic acid bacteria at a pH ranging from 6.0 to 7.0 at a temperature ranging from 25° C. to 35° C. for a time sufficient to grow the Nisin-producing lactic acid bacteria; and
selecting Nisin-producing lactic acid bacteria which produce 6,800 IU or more of Nisin per milliliter of a supernatant of a medium in batch culturing with a liquid medium.

13. The method as claimed in claim 12, wherein after said growing said method further comprises
inducing spontaneous mutation or mutation with a mutation inducer or ultraviolet rays to produce a mutant strain;
plating the mutant strain on synthetic agar medium containing one or more bacteriocin; and
growing said mutant strain on said synthetic agar medium for a time and under conditions suitable to grow said mutant strain.

14. The method as claimed in claim 12, wherein the bacteriocin is Enterocin or Nisin.

15. The method as claimed in claim 12, wherein the bacteriocin is Nisin in an amount of from 11,000 to 90,000 IU per milliliter of the medium.

16. The method as claimed in claim 12, wherein the bacteriocin is Nisin in an amount of from 20,000 to 80,000 IU per milliliter of the medium.

17. A culture solution containing lactic acid bacteria, which is obtained by batch culturing in a liquid medium a lactic acid bacteria which produce 6,800 IU or more of Nisin per milliliter of a supernatant of the medium.

18. A food, drink, or feed comprising the 0.01 to 10% of the culture solution according to claim 17.

19. A dry product of the culture solution of claim 17, wherein said culture solution is spray-dried or drum-dried.

20. A food, drink, or feed comprising the 0.01 to 10% of the dry product of the culture solution according to claim 19.

21. A supernatant of the culture solution of claim 17, wherein said lactic acid bacteria are removed from said culture solution.

22. A food, drink, or feed comprising the 0.01 to 10% of the supernatant of the culture solution according to claim 21.

23. A dry product of the supernatant of the culture solution according to claim 21, wherein said supernatant is spray-dried or drum-dried.

24. A food, drink, or feed comprising the 0.01 to 10% of the supernatant of the culture solution according to claim 23.
